(19) United States
(12) Patent Application Publication
(10) Pub. No.: US 2017/0152293 A1
(43) Pub. Date: Jun. 1, 2017

(54) RECOMBINANT CYTOTOXIN AND USE THEREOF

(71) Applicant: NATIONAL YANG MING UNIVERSITY, Taipei City (TW)

(72) Inventors: Mao-Jung LIN, Taipei City (TW); Wen-Liang LO, Taipei City (TW); Tao-Tien CHEN, New Taipei City (TW)

(21) Appl. No.: 15/219,496

(22) Filed: Jul. 26, 2016

(30) Foreign Application Priority Data
Nov. 26, 2015 (TW) 104139300

(51) Int. Cl. C07K 14/38

(52) U.S. Cl. CPC C07K 14/38; C07K 2319/10; A61K 38/00

(57) ABSTRACT

A recombinant cytotoxin is provided. The recombinant cytotoxin of the present invention comprises a cytotoxin, a cell penetrating peptide (CPP), and Asp-Glu-Val-Asp (DEVD) sequence inserted in the cytotoxin. The recombinant cytotoxin can induce a targeting cell into the apoptotic pathway and be cleaved by the enzyme generated from apoptotic pathway. The present invention also provides a method for treating cancer, comprising administrating the recombinant cytotoxin to a subject.

### CROSS-REFERENCE TO RELATED APPLICATIONS

This Non-provisional application claims priority under 35 U.S.C. §119(a) on Patent Application No(s). 104139300 filed in Taiwan, Republic of China Nov. 26, 2015, the entire contents of which are hereby incorporated by reference.

### FIELD OF THE INVENTION

The present invention relates to a recombinant cytotoxin, and especially relates to a programmed self-destruct cytotoxin, and particularly relates to a cytotoxin can result in apoptotic activity towards targeted cells and then the cytotoxin can be hydrolyzed and destroying itself.

### BACKGROUND OF THE INVENTION

A toxin is an organic or inorganic substance which, even at low concentrations, has a deleterious effect on living organisms. Many bacteria and higher plants produce cytotoxic proteins collectively called ribotoxins which function by being taken up by, and then inactivating the ribosomes of a target cell. The ribotoxins are considered to fall into two major classes: (1) NAD+-dependent ribotoxins, which appear to disable ribosomes by covalently attaching ADP-ribose to “elongation factor-2” protein; and (2) NAD+-independent ribotoxins, which appear to inactivate the 60S ribosomal subunit. It is the NAD+-independent ribotoxins and their derivatives to which the separation and purification methods of the invention apply. These ribotoxins affect only eucaryotic ribosomes which are lethal at low concentrations.

A ribotoxin may be a heterodimer or a polypeptide, wherein the heterodimer is formed by an enzymatically A chain polypeptide and a enzymatically inactive B chain polypeptide linked by disulfide bonds, and the non-catalytic B chain polypeptide binds to surface of a target cell to stimulate uptake of the ribotoxin into the cell. The heterodimer ribotoxin includes ricin, abrin, and modeccin. Other ribotoxins are single polypeptides which are cytoloxioally active, and are thus sometimes referred to as “A chain toxins” or “hemitoxins”.

Several ribotoxins, such as ricin and abrin, occur in nature in more than one form. Thus, these ribotoxins can be considered to represent several isotoxins, i.e. structurally similar proteins with quantitatively differing functional properties.

Some attempts have been made to take advantage of the cytotoxic properties of the ribotoxins by employing the unmodified polypeptides as therapeutic agents. However most efforts to use ribotoxins therapeutically have been focused on hybrid toxins, in which the cytotoxic moiety is covalently coupled to a “binding moiety” expected to bind specifically to certain cells, virus, or other macromolecules. The most common examples of hybrid toxins are immunotoxins, wherein the cytotoxic polypeptide is conjugated to a specific antibody; however, a variety of other binding moieties may be used. However, the hybrid toxins still would affect other normal periphery cells even if they have specificity.

### SUMMARY OF THE INVENTION

In view of the above-mentioned problem, the present invention provides a novel recombinant cytotoxin. The recombinant cytotoxin of the present invention with apoptosis activity is able to kill target cells and does not affect the cells surrounding the target cells.

The present invention provides a recombinant cytotoxin comprising a cytotoxin, a penetrating peptide, and an Asp-Glu-Val-Asp (DEVD) sequence, wherein the DEVD sequence is inserted into the cytotoxin.

In one embodiment, the penetrating peptide is linked to the cytotoxin through a linker.

In one embodiment, the cytotoxin comprises ribotoxin, snake venom, bee venom, jellyfish venom or toad venom.

In one embodiment, the ribotoxin is selected from a group consisting of fungal-originated ribotoxin, ricin, abrin, emetine, diphtheria toxin, cinnamomin, camphorin.

In one embodiment, the fungal-originated ribotoxin comprises α-sarcin, gigantin, mitogllin, restrictocin, allergen, clavin or tricholin.

In one embodiment, the penetrating peptide comprises Tat, antennapedia or polyarginine.

In one embodiment, the DEVD sequence is inserted into the loop region of the ribotoxin.

In one embodiment, the DEVD sequence is inserted into the loop 2 of the ribotoxin.

In one embodiment, the Tat is inserted into the N-terminus of the cytotoxin.

The present invention also provides a pharmaceutical composition comprising the recombinant cytotoxin of the present invention and a pharmaceutically acceptable carrier.

The present invention further provides a use of the recombination cytotoxin of the present invention for preparing a pharmaceutical composition of treatment of cell proliferation disease.

In one embodiment, the recombinant cytotoxin is toptically administrated.

In one embodiment, the proliferation disease is cancer.

In one embodiment, the cancer comprises oral cancer, breast cancer, prostate cancer, leukemia, colorectal cancer, uterine cancer, ovarian cancer, endometrial cancer, cervical cancer, testicular cancer, lymphoma, rhabdomyosarcoma, neuroblastoma, pancreatic cancer, lung cancer, brain tumors, skin cancer, stomach cancer, liver cancer, kidney cancer or nasopharyngeal cancer.

Detailed description of the invention is given in the following embodiments with reference to the accompanying drawings.

### BRIEF DESCRIPTION OF THE DRAWINGS

FIG. 1A illustrates a design of the recombinant cytotoxin according to an embodiment of the invention.

FIG. 1B illustrates another design of the recombinant cytotoxin according to another embodiment of the invention.

FIG. 2A-2E illustrate that the recombinant cytotoxin of the present invention stimulate a cell into apoptosis pathway.

FIG. 3A-3B is an image of SDS-PAGE electrophoresis gel. FIG. 3A-3B illustrates that the KZ-sarcin is hydrolyzed by caspase-3 to two fragments of 12 kb and 8.5 kb.

FIG. 4 is an image of RNA gel electrophoresis. FIG. 4 illustrates that 28S rRNA is hydrolyzed by KZ-sarcin protein to a fragment. The results demonstrate the RNA-hydrolytic activity of KZ-sarcin in vitro.

FIG. 5 illustrates that KZ-sarcin significantly suppresses the synthesis of proteins. The decrease of protein translation efficiency is dependent upon increasing KZ-sarcin concentration.

FIGS. 6A-6D are images of fluorescence (FIG. 6A and FIG. 6C) or phase contrast microscope. FIG. 6B is shown a merge photograph consisting of phase contrast photograph and FIG. 6A. FIG. 6D is shown a merge photograph consisting of phase contrast photograph and FIG. 6C. The fluorescent signals are detected in the cells treated with KZ-sarcin (FIG. 6C). The results indicate that the KZ-sarcine can enter cells (FIG. 6C and FIG. 6D).

FIG. 7 illustrates KZ-sarcin can inhibit the synthesis of proteins. The inhibition of protein synthesis is dependent upon increasing treatment time of KZ-sarcin.

FIGS. 8A and 8B are images of fluorescence microscope. The cells form vacuoles and are degraded after treatment of KZ-sarcin (FIG. 8A). KZ-sarcin induces the apoptosis of cells. The cells do not form vacuoles and are not degraded after treatment of α-sarcin (FIG. 8B).

FIG. 9 illustrates an 8.5 kD fragment (C-terminal peptide) is produced in cells after treatment of KZ-sarcin for 2 hours.

FIG. 10 illustrates the activity of caspase-3 after treatment of KZ-sarcin for 1, 2, 3, 4 and 5 hours, respectively.

FIG. 11 illustrate that KZ-sarcin significantly suppress or even stop the tumor growth in animal studies.

FIGS. 12A-12D also illustrate that KZ-sarcin significantly suppress or even stop the tumor growth in animal studies. FIG. 12A is an image showing mice were injected daily for Day 1 with 10 ul of PBS. FIG. 12C is an image showing mice were injected daily for Day 1 with KZ-sarcin.

FIG. 13A-D is an image showing H&E-stained slides. FIG. 13A-B illustrates that chromatin condensation, multiple nuclear fragmentation and apoptotic body are formed after KZ-sarcin treatment. Alternately, FIG. 13C-D are images of cells without KZ-sarcin treatment maintain the original shape.

FIG. 14A is a computer graphic showing the 3-dimensional structure of KZ-sarcin. FIG. 14B is a computer graphic showing the 3-dimensional structure of wild type α-sarcin.

### DETAILED DESCRIPTION OF THE INVENTION

The present disclosure provides novel fusion proteins. Various aspects of the present disclosure relate to fusion proteins, compositions thereof, and methods for making and using the disclosed fusion proteins. By administrating the novel fusion proteins of the present invention to an organism, significantly increased positive response can be seen within the organism.

The following is a detailed description provided to aid those skilled in the art in practicing the present invention. Those of ordinary skill in the art would understand that modifications or variations of the embodiments expressly described herein, which do not depart from the spirit or scope of the information contained herein, are encompassed by the present disclosure. The terminology used in the description is for describing particular embodiments only and is not intended to be limiting of the invention. The section headings used below are for organizational purposes only and are not to be construed as limiting the subject matter described.

The present invention provides a recombinant cytotoxin, a penetrating peptide, and an Asp-Glu-Val-Asp (DEVD) sequence, wherein the DEVD sequence is inserted into the cytotoxin.

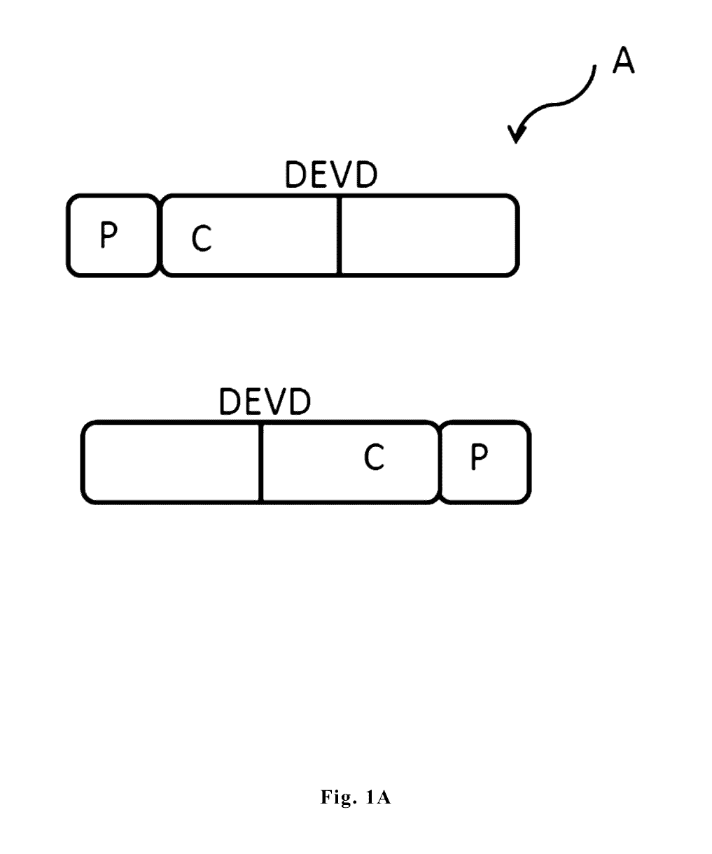

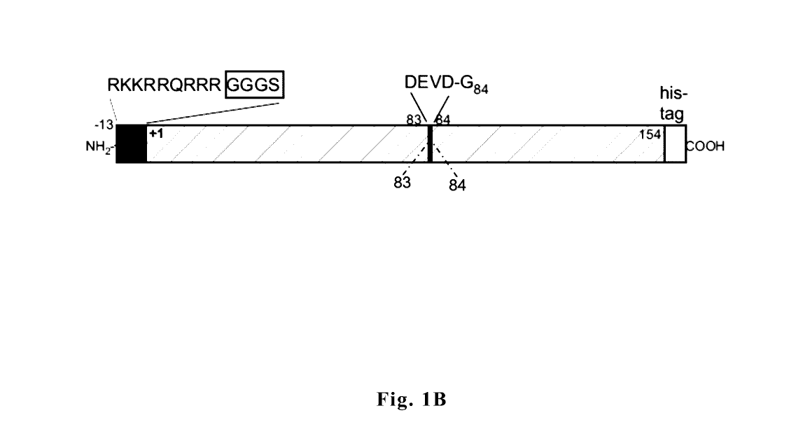

Referring to FIG. 1A, the recombinant cytotoxin A of the present invention comprises cytotoxin C and cell penetrating peptide P. Cell penetrating peptide P can be located at the N-termunus or C-terminus of cytotoxin C. Cell penetrating peptide P can be linked to cytotoxin C via one or more linker(s). DEVD sequence is a short peptide “Asp-Glu-Val-Asp”, which can be recognized by caspase-3. DEVD sequence is inserted into the cytotoxin. It shall be noted that DEVD sequence does not affect the activity of cytotoxin. Therefore, the recombinant cytotoxin of the present invention still has the cytotoxin activity.

The term “toxin” as used herein, generally refers to specific, characterizable, poisonous chemicals, often proteins, with specific biological properties, including immunogenicity, produced by microbes, higher plants or animals.

The toxin includes any toxin substances produced by any living organisms (including bacteria and plants). The toxin includes, but is not limited to, ribotoxin, snake venom, bee venom, jellyfish venom or toad venom, preferably ribotoxin. The recombinant cytotoxin of the present invention can be produced using DNA recombinant technology. The cytotoxin can be covalently linked with another functional protein.

The term “ribotoxin” as used herein, generally refers to any peptide or polypeptide produced naturally or synthetically which is capable of targeting and enzymatically releasing a specific base located within a specific base sequence in a nucleic acid substrate. The ribotoxin includes, but is not limited to, fungal-originated ribotoxin, ricin, abrin, emetine, diphtheria toxin, cinnamomin and camphorin.

Further, the fungal-originated ribotoxin includes, but is not limited to, α-sarcin, gigantic, mitogllin, restrictocin, allergen, clavin and tricholin, preferably α-sarcin.

The term “cell penetrating peptide (CPP)” as used herein, generally refers to carrier peptide that is capable of crossing biological membrane or a physiological barrier. Cell penetrating peptides are also called cell-permeable peptides, protein-transduction domains (PTD) or membrane-translocation sequences (MTS). CPPs have the ability to translocate in vitro and/or in vivo the mammalian cell membranes and enter into cells, and directly carries an interestingly conjugated compound, such as a drug or marker, to a desired cellular destination, e.g. into the cytoplasm (cytosol, endoplasmic reticulum, Golgi apparatus, etc.) or the nucleus. Accordingly, the CPP can direct or facilitate penetration of an interesting compound across a phospholipid, mitochondrial, endosomal or nuclear membrane. The CPP can also directly carry an interesting compound from outside the cell through the plasma membrane, and into the cytoplasm or to a desired location within the cell, e.g., the nucleus, the ribosome, the mitochondria, the endoplasmic reticulum, a lysosome, or a peroxisome. Alternatively or in addition, the CPP can directly carry an interesting compound across the blood-brain, trans-mucosal, hematoretinal, skin, gastrointestinal and/or pulmonary barriers.

Several proteins and their peptide derivatives have been found to possess cell internalization properties including but not limited to the Human Immunodeficency Virus type 1 (HIV-1) protein Tat (Ruben et al. J. Virol. 63, 1-8 (1989)), the herpes virus tegument protein VP22 (Elliott and O'Hare, Cell 88, 223-233 (1997)), the homeotic protein of Drosophila melanogaster Antennapedia (the CPP is called Penetratin) (Derossi et al., J. Biol. Chem. 271, 18188-18193 (1996)), the protegrin 1 (PG-1) anti-microbial peptide SynB (Kokryakov et al., FEBS Lett. 327, 231-236 (1993)) and the basic fibroblast growth factor (Jans, Faseb J. 8, 841-847 (1994)). A number of other proteins and their peptide derivatives have been found to possess similar cell internalization properties. The carrier peptides that have been derived from these proteins show little sequence homology with each other, but are all highly cationic and arginine or lysine rich. Indeed, synthetic poly-arginine peptides have been shown to be internalized with a high level of efficiency (Futaki et al., J. Mol. Recognit. 16, 260-264 (2003); Suzuki et al., J. Biol. Chem. (2001)).

The term “linker” as used herein, generally refers to a covalent bond, preferably a peptide bond. The recombinant cytotoxin may optionally include at least one linker. The linker is between the cytotoxin and cell penetrating peptide (CPP). In one embodiment, the linker comprises 1 to 5 amino acids.

In a specific embodiment, the cytotoxin is α-sarcin, cell penetrating peptide is Tat, and DEVD sequence is located at the loop region of α-sarcin, preferably loop 2 region, wherein the DEVD sequence more preferably is located at amino acid position 84 (Gly) of α-sarcin (FIG. 1B).

The present invention further provides a pharmaceutical composition comprising the recombinant cytotoxin and a pharmaceutically acceptable carrier.

The composition for treatment is formulated to be compatible with the route of administration. The composition can be formulated as a powder, a tablet, a pill, a granule, a capsule, a lotion, a suspension, a liposome formulation, a nasosphere, a patch, a suppository, an enema, a drip infusion, or an injection solution. The composition can be administered orally, intraarticularly, intraperitoneally, intrathecally, intrarterially, intranasally, intraparenchymally, subcutaneously, intramuscularly, intravenously, dermally, intrarectally, or topically.

The term “subject” as used herein, generally refers to human or non-human mammal, e.g. a dog, a cat, a mouse, a rat, a cow, a sheep, a pig, a goat, or a primate, and expressly includes laboratory mammals, livestock, and domestic mammals. In one embodiment, the mammal may be a human; in others, the mammal may be a rodent, such as a mouse or a rat. In another embodiment, the subject is an animal model (e.g., a transgenic mouse model).

A solution for parenteral, intradermal, or subcutaneous administration can include: a sterile diluent such as water, saline, glycerin, fixed oils, polyethylene glycols, propylene glycol, or other synthetic solvents; an antibacterial agent such as benzyl alcohol or methyl parabens; an antioxidant such as ascorbic acid or sodium bisulfite; a chelating agent; or a buffering agent such as acetate or phosphate. The solution can be stored in ampoules, disposable syringes, or plastic or glass vials.

A formulation for injection or intravenous administration can include a carrier which is a solvent or a dispersion medium. Suitable carriers include water, physiological saline, bacteriostatic water, Cremophor EL™ (BASF, Parsippany, N.J.) phosphate buffered saline (PBS), ethanol, polyols (e.g., glycerol, glycol, propylene glycol, and the like), and mixtures thereof. These compositions must be steriled and liquefied for injection. Fluidity of these compositions can be maintained with, for example but not limited, lecithin or a surfactant. Microbial contamination can be prevented by the inclusion of antibacterial and antifungal agents, such as parabens, chlorobutanol, phenol, ascorbic acid, and thimerosal. Sugars and polyalcohols, such as manitol, sorbitol or sodium chloride, can be used to maintain isotonicity in the composition.

The present invention further provides a use of the recombinant cytotoxin for preparing a pharmaceutical composition of treatment of cell proliferation disease.

The recombinant cytotoxin of the present invention can pass across the membrane and penetrate into the cells to kill the cells. Next, the enzymes generated from apoptosis, such as caspase-3, can recognize the DEVD sequence on the recombinant cytotoxin and destroy the structure of the cytotoxin.

The term “cell proliferative disorders” as used herein, generally refers to disorders wherein unwanted cell proliferation of one or more subset(s) of cells in a multicellular organism occurs, resulting in harm to the multicellular organism. Cell proliferative disorders can occur in different types of animals and in humans. Cell proliferative disorders include, but are not limited to, cancers, blood vessel proliferative disorders, and fibrotic disorders, preferably cancer. The cancer includes, not is not limited to, oral cancer, breast cancer, prostate cancer, leukemia, colorectal cancer, uterine cancer, ovarian cancer, endometrial cancer, cervical cancer, testicular cancer, lymphoma, rhabdomyosarcoma, neuroblastoma, pancreatic cancer, lung cancer, brain tumors, skin cancer, stomach cancer, liver cancer, kidney cancer or nasopharyngeal cancer, preferably oral cancer.

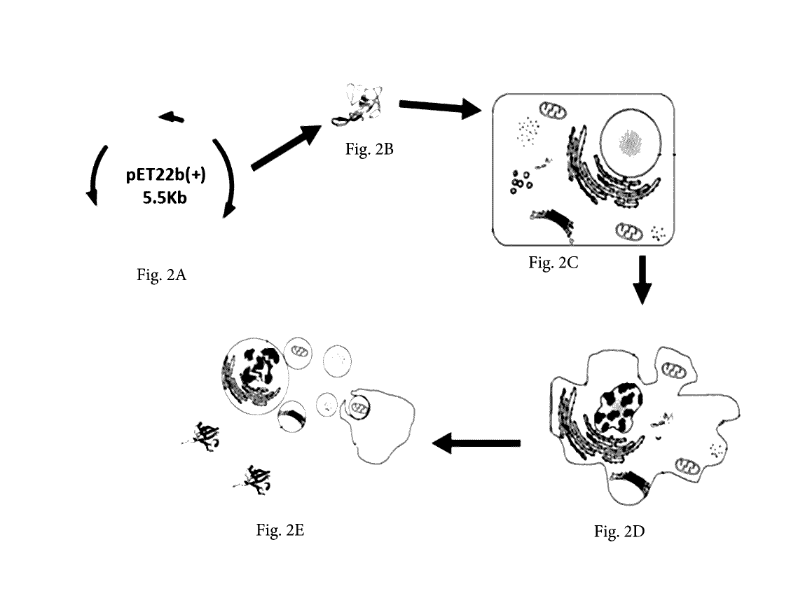

FIG. 2A-2E illustrate the cytotoxic process of the recombinant cytotoxin of the present invention. Referring to FIG. 2A-2B, a vector is provided to express a recombinant cytotoxin. As shown in FIG. 2C, the recombinant cytotoxin contacts with a cell. The recombinant cytotoxin can transport across the membrane and into cells via cell penetrating peptide (CPP). The recombinant cytotoxin can stimulate the cell to undergo the apoptotic pathway (FIG. 2D) and generate caspase-3. Caspase-3 can recognize the DEVD sequence on the recombinant cytotoxin to cleave the recombinant cytotoxin (FIG. 2E).

The recombinant cytotoxin can penetrate into the cell which stimulated to undergo the apoptotic pathway and cleaved by the enzyme generated from apoptotic pathway. Thus, the recombinant cytotoxin specifically affects the target cell, and does not affect the cells surrounding the target cell.

Additional specific embodiments of the present invention include, but are not limited to the following:

### Example 1

### Construction of Recombinant Cytotoxin

The gene fragment of α-sarcin protein was amplified from filamentous fungus Aspergillus giganteus using polymerase chain reaction (PCR). The gene fragment was ligated into pET22b plasmid to make a KZ-sarcin plasmid. Tat peptide was fused in the N-terminus of α-sarcin using pET28a/α-sarcin as a template for PCR amplification with two N-primers and C-primer. The N1-primer was 5′-NNNCCATGGGTAGAAAAAAACGAAGACAACGACGAAGAGGTGGTGGTAGC-3′ (SEQ ID NO: 1). The N2-primer was 5′-GACGAAGAGGTGGTGGTAGC gt-gacctggacctgcttgaacg-3′ (SEQ ID NO: 2). C-primer was

(SEQ ID NO: 3)
5′-TAAAGCGGCCGCAtgagagcagagettaagttc-3′.

N1 primer carried the basic domain sequence of Tat peptide (MGRKKRRQRRR (SEQ ID NO: 4)) with linker (GGGS (SEQ ID NO: 5)) and N_{CO} I site (underlined), N2 primer carried overlapping sequence of N1-primer (uppercase) and α-sarcin specific sequence (lowercase), and C-primer carried α-sarcin specific sequence (lowercase) with a N_{OT} I site (underlined). After PCR amplification, the PCR products were digested by N_{CO} I and N_{OT} I restriction enzyme and ligated into pET22b plasmid to make a pET-22b/Tat-sarcin plasmid.

In the second-step of construction, two primers, the upper and lower primers, were used to insert a DEVD sequence (SEQ ID NO: 8) into the loop 2 of α-sarcin for amplification with pET-22b/Tat-sarcin plasmid as template. The upper primer was 5′-GACGAAGTGGATggcaagagtgatcactacctgctggag-3′ (SEQ ID NO: 6), carries a coding sequence of DEVD (uppercase) with corresponding sequence of α-sarcin (lowercase). The lower primer was 5′-cttgctgtgcttgggaggacg-3′ (SEQ ID NO: 7), carries α-sarcin specific sequence (lowercase) with corresponding sequence of α-sarcin. After PCR amplification, the PCR products were purified, self-ligated and transformed into the competent cells ECOS101 to obtain the pET22b/KZ-sarcin (pET-22b/Kazecin) plasmid.

### Example 2

### Expression and Purification of Recombinant Protein

Recombinant pET22b/KZ-sarcin plasmid was expressed in E. coli strain BL21 CodonPlu (DE3) in LB broth under IPTG induction at 37° C. for 2 hours. The culture medium was centrifuged to obtain bacteria pellet. The bacteria pellet was added to 50 mL lysis buffer and lysed by a sonicator. After high speed centrifugation at 39,000 g for 1 hour, the supernatant was removed to collect inclusion bodies. The inclusion bodies were lysed in denature binding buffer by sonicator. After high speed centrifugation, the supernatant was reacted with Ni+-His resin (Novagen) for 2 hours, washed with denature wash buffer and eluted with denature elute buffer to obtain KZ-sarcin recombinant protein (SEQ ID NO: 9). The KZ-sarcin recombinant protein (Kazecin) has the sequences as follows.

M K Y L L P T A A A G L L L L
A A Q P A M A M G R K K R R Q
R R R G G G S V T W T C L N D
Q K N P K T N K Y E T K R L L
C N Q N K A E S N S H H A P L
S D G K T G S S Y P H W F T N
G Y D G E G K I L K G R T P I
K F G K S D C D R P P K H S K
D E V D G K S D H Y L L E F P
T F P D G H D Y K F D S K K P
K E D P G P A R V I Y T Y P N
K V F C G I I A H T K E N Q G
E L K L C S H A A A L E H H H
H H H

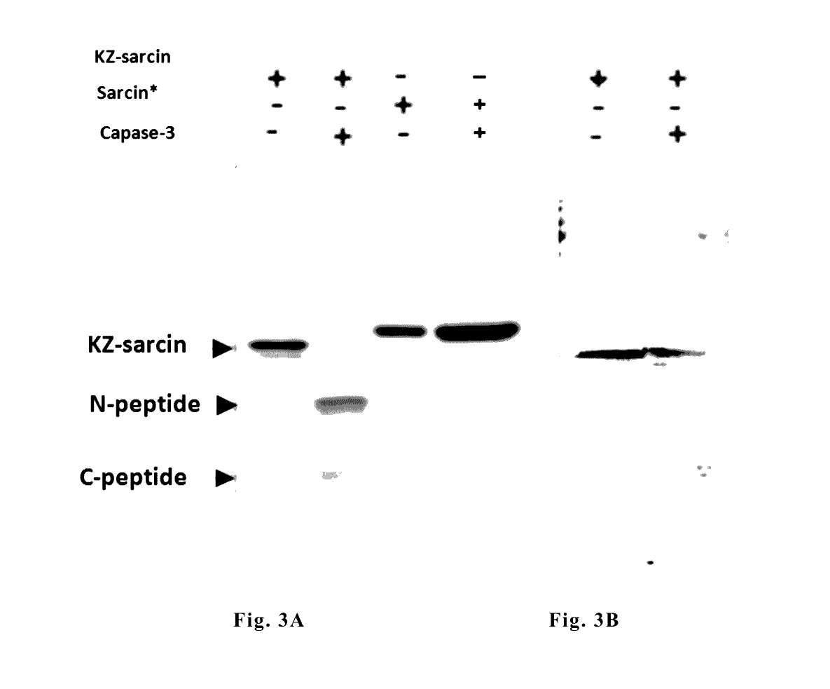

The KZ-sarcin recombinant protein (Kazecin) was confirmed by SDS-PAGE. KZ-sarcin was treated with caspase-3 (Sigma Chem. Co, U.S.A.) and PBS buffer at room temperature for 15 minutes, respectively, and then analyzed by SDS-PAGE. In control group, a mutant sarcin (Sarcin*) was used. As shown in FIG. 3A-3B, KZ-sarcin recombinant protein was hydrolyzed by caspase-3 into two fragments of 12 kb and 8.5 kb.

### Example 3

### Ribosome Inactivation Assay

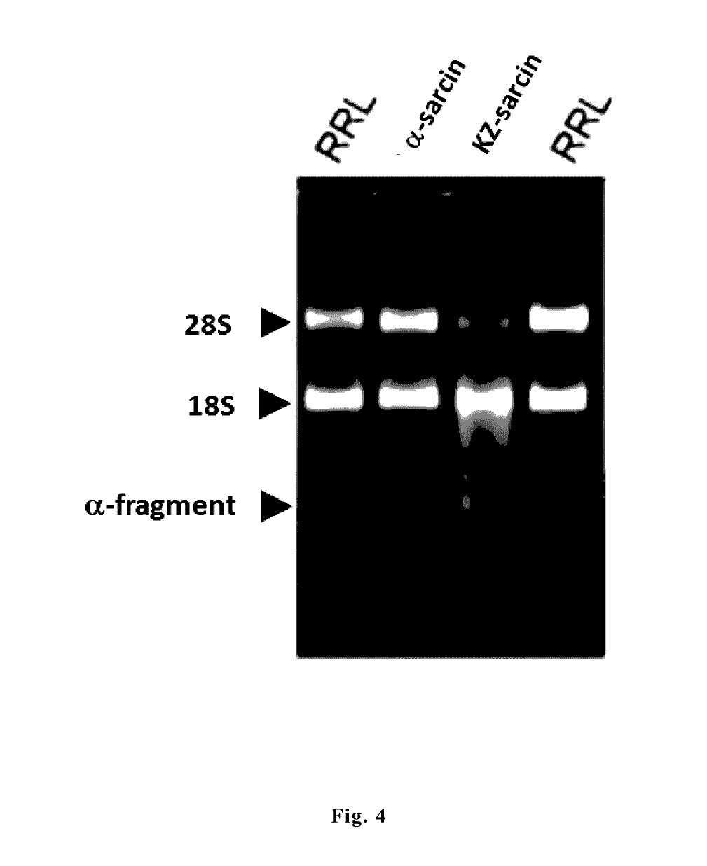

The rabbit reticulum lysate (RRL) was used in this Example to proceed to ribosome inactivation assay. The rabbit reticulum lysates (Promega Co.) were treated with the KZ-sarcin and analyzed by 1% agarose gel electrophoresis. Referring to FIG. 4, 28s RNA was hydrolyzed to form a fragment. The results indicate that KZ-sarcin and wild type α-sarcin both had the RNA hydrolysis activity. Further, the hydrolysis activity of KZ-sarcin was not destroyed even if Tat and DEVD peptide were inserted.

### Example 4

### In Vitro Cell Free System Protein Synthesis Assay

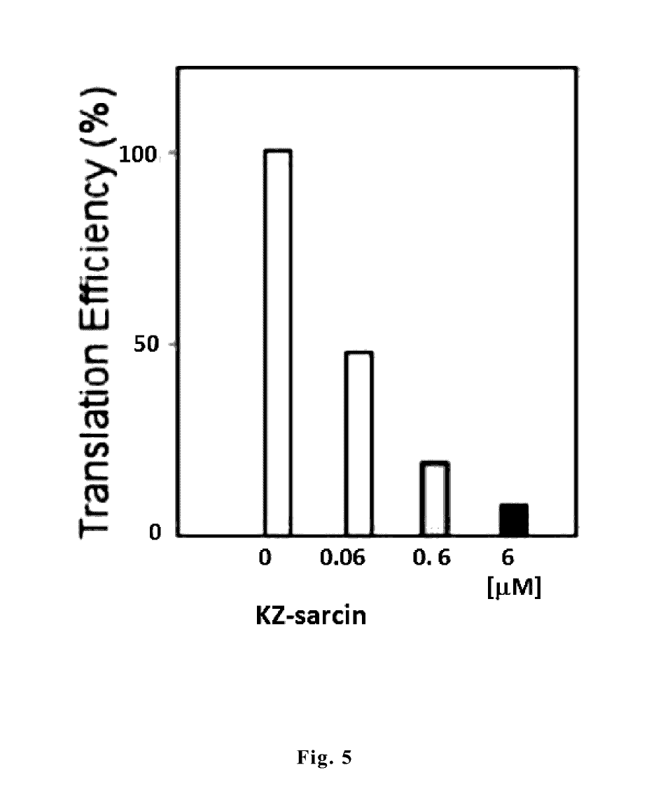

RRL translation system was used in this Example. The RRL was treated with KZ-sarcin at different concentration in solution containing 20 mM Hepes, 5 mM dithiothreitol, 5 mM magnesium acetate, 100 mM potassium acetate, 2 mM ATP, 0.4 mM GTP, 8 mM creatine phosphate, 50 mg/mL creatine phosphokinase, plus 20 μM amino acid mixture minus methionine, and 1200 Ci/mmol at 1 mCi/mL [³⁵S]methionine. The cellular translation was initiated by additional 40 μg/mL luciferase mRNA at 37° C. for 90 min. The [³⁵S] incorporated protein was TCA-precipitated, and collected by GF/A glass filter (Whatman Co.). The incorporation of [³⁵S] radioactivity was counted in a liquid scintillation counter (Tri-Carb 2900TR). As shown in FIG. 5, the protein translation efficiency was decreased dependent upon increasing the concentration of KZ-sarcin. The results indicate that KZ-sarcin significantly suppressed the synthesis of proteins.

### Example 5

### Activity of KZ-Sarcin into Cells

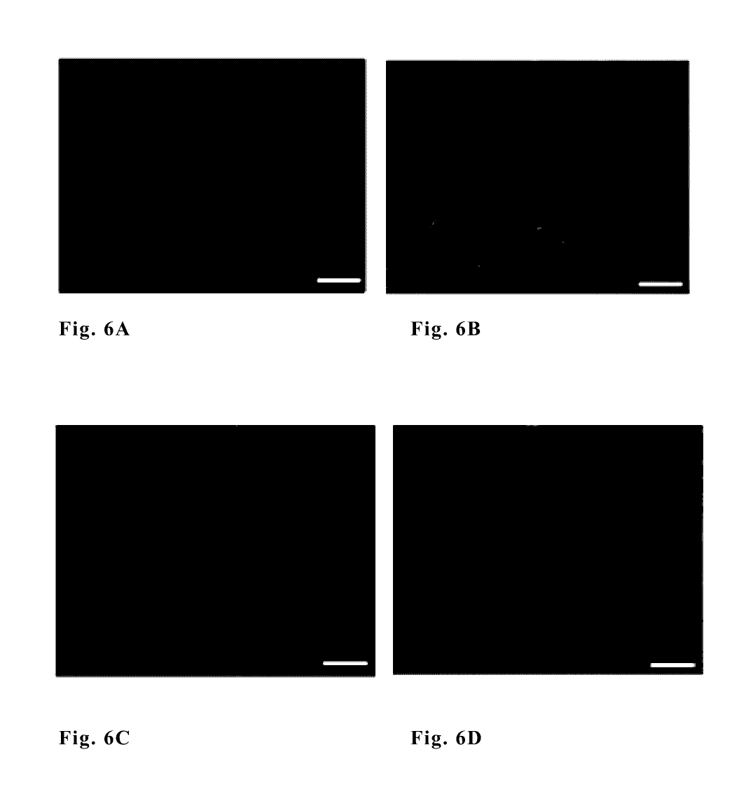

KZ-sarcin or α-sarcin was chemically conjugated with fluophore Alexa-555. This was carried out by mixing 500 μg of KZ-sarcin or α-sarcin with 50 μg of fluophore Alexa-555 (Invitrogen Co.) in PBS to a final volume of 500 μL. HeLa cells were treated with serum free DMEM containing 2 μL of KZ-sarcin or α-sarcin at 37° C. for 1 hr. The mixture was washed with PBS solution and then observed by fluorescent microscopy and phase contrast microscopy. As shown in FIG. 6A-6D, the positively fluorescent signals could be detected and located within the HeLa cells via treated by KZ-sarcin after 1 hour (FIG. 6C-6D).

In contrast, no fluorescent signal was observed in the cells that had been treated with fluorescent-labeled wild type α-sarcin (FIG. 6A-6B). The results indicate that KZ-sarcin can directly enter the cells.

### Example 6

### Ex Vivo Inhibition of Protein Synthesis Assay

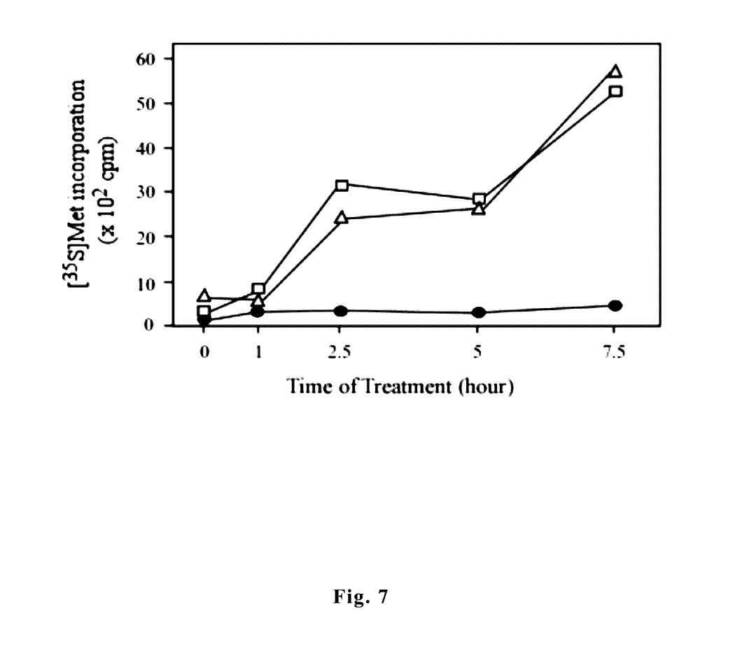

293T cells (OD₆₅₀=0.3) were grown in 50-mL flask in the presence of [³⁵S]methionine (the final concentration of 3 mCi/mL; Amersham, U.S.A.). When the cell culture had attained 0.5 OD₆₅₀ units, cell culture was treated with recombinant KZ-sarcin (1 μM). At various time intervals, the cells were taken and lysed immediately in 1 mL of ice-cold trichloroacetic acid (TCA). Peptides that incorporated with [³⁵S]methionine were collected on Whatman GF/C filters. The incorporation of [³⁵S] radioactivity was counted by scintillation counter (Tri-Carb 2900TR). In FIG. 7, () represents cell treated with KZ-sarcin, (□) represents cell treated with α-sarcin, and (Δ) represents control group. The protein synthesis was significantly inhibited after treatment of KZ-sarcin. The inhibition of protein synthesis was increased dependent upon increasing the treatment time.

### Example 7

### Hoechst 33342 Staining Assay

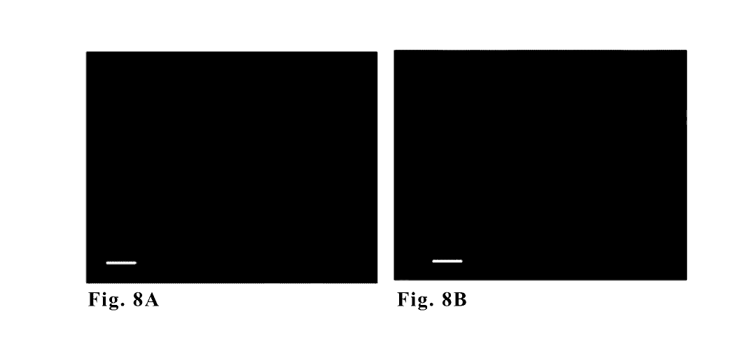

HeLa cells grown on glass cover slides were washed twice with PBS and treated with serum free DMEM containing 2 μM of α-sarcin or KZ-sarcin (Kazecin) at 37° C. for 1 hour. After washing cells twice with PBS, the cells were incubated with Hoechst 33342 in serum free DMEM at 37° C. for 1 hour. At the removing the dye, the cells were fixed with 4% paraformaldehyde and visualized by fluorescent microscopy (Olympus FV1000). As shown in FIG. 8, the treated cells produced numerous vacuoles and the nuclei were cleaved to form fragmented nuclei (FIG. 8A). The results indicate that KZ-sarcin induced cells to lead to apotosis.

### Example 8

### Ex Vivo Responses of KZ-Sarcin to Caspase-3 Activity

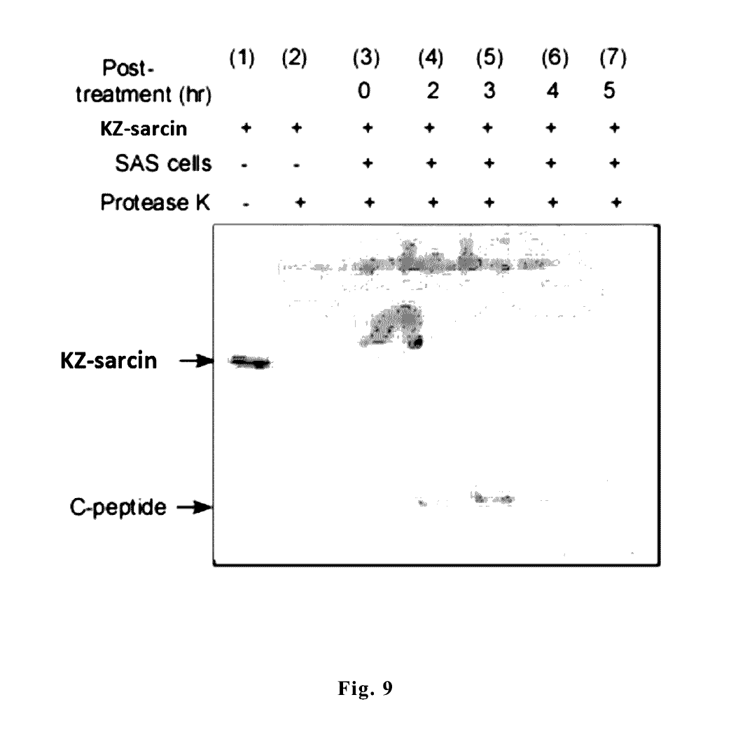

Oral SAS cells were treated with KZ-sarcin and then collected at different time intervals of 1, 2, 3, 4 and 5 hours post-incubation. The cells were treated with trypsin at 25° C. for 30 minutes. After several thoroughly washings, cells were lysed with denaturation solution containing 1% SDS and analyzed on a SDS-PAGE, followed by western blotting analysis using anti-His antibody. FIG. 9 shows western blot of the KZ-sarcin (column 1), KZ-sarcin treated with trypsin (column 2), and cells (1×10⁶) treated with KZ-sarcin at different time (columns 3 to 7). After 2 hours of KZ-sarcin treatment, cells produced a peptide fragment of 8.5 kD (C-terminal peptide). A similar phenomenon can be found until 5 hours after treatment.

### Example 9

### Assay of Caspase-3 Activity

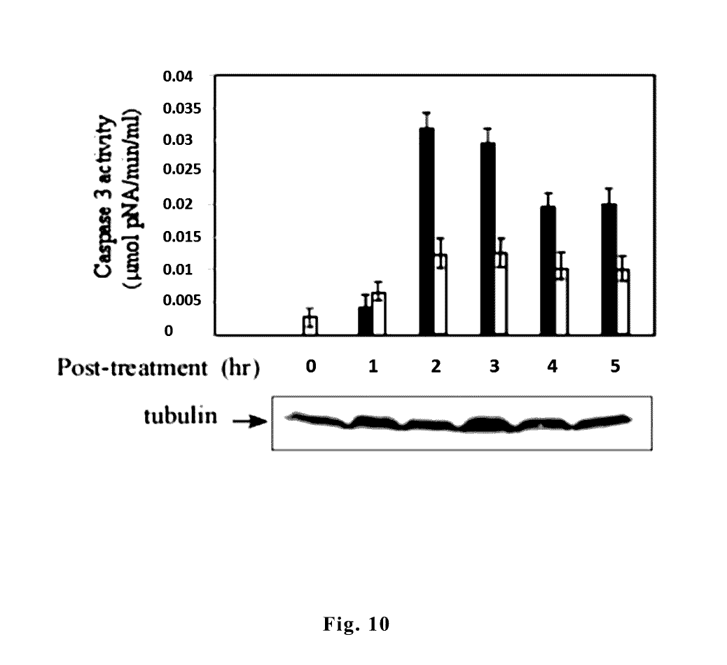

The SAS cells collected in Example 8 were lysed by a lysis buffer (50 mM HEPES, pH7.4, 25 mM CHAPS, 25 mM DTT). The cell lysates were incubated with caspase-3 substrate (Ac-DEVD-pNA) at 37° C. for overnight. The caspase-3 activity was measured at the absorbance at 405 nm using Ultrospec 3300 pro (Amersham Biosciences). FIG. 10 shows the activity of caspase-3 in cells at different time. Referring to FIG. 9 and FIG. 10, the activity of caspase-3 was corresponded to the production of 8.5 kD of fragment (C-terminal peptide).

### Example 10

### Animal Study

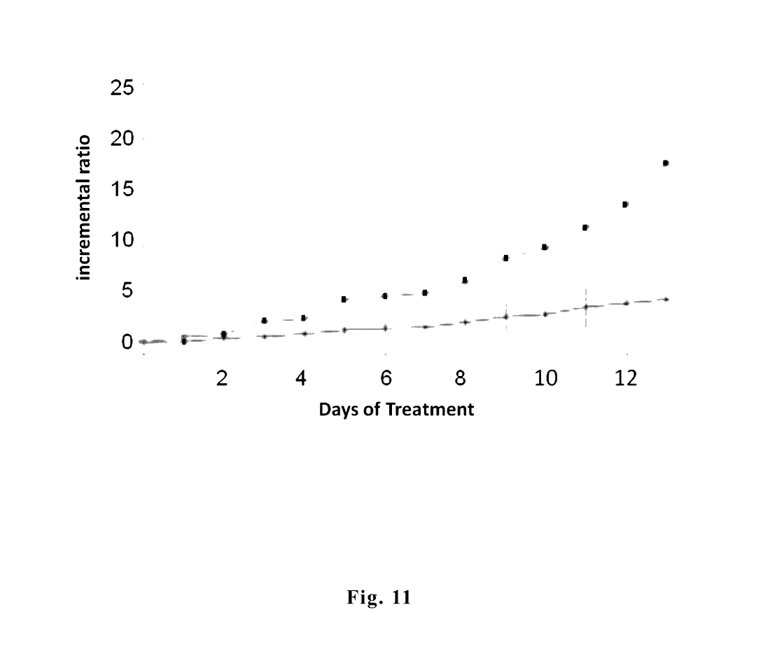

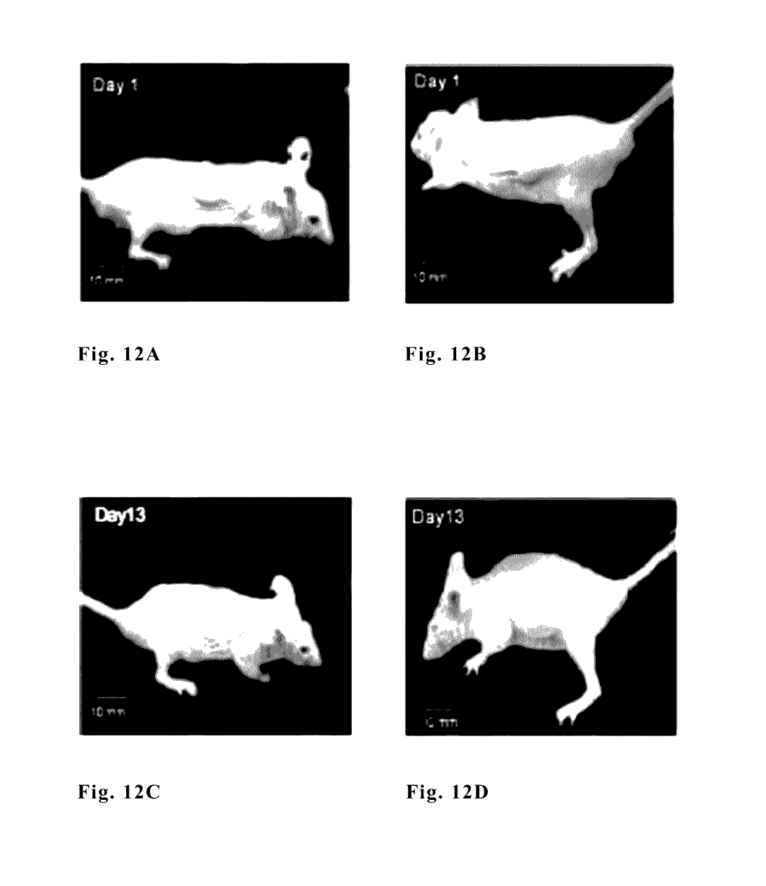

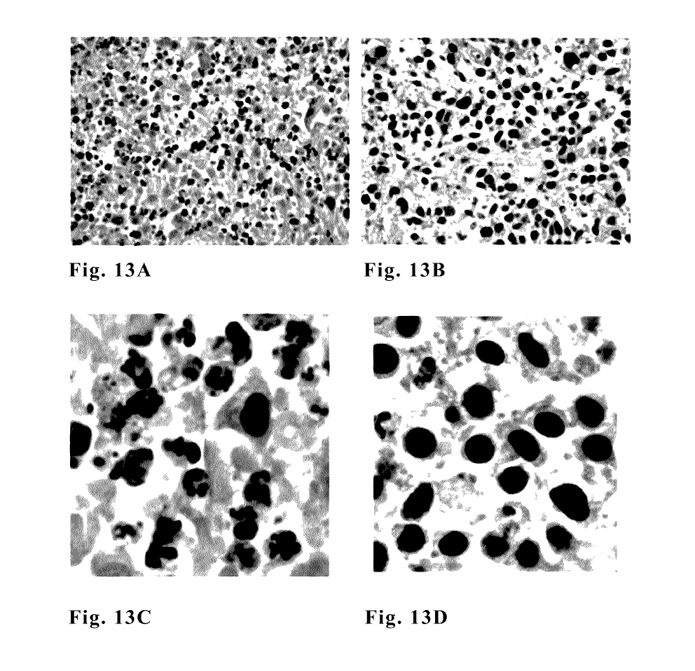

In this Example, as shown in FIG. 11 and FIG. 12, Eight-week-old male nude mice (BALB/cAnN-Foxn1; NLAC, Taipei, Taiwan) were utilized for in vivo experiments. Mice were injected with 2×10⁶ cells of the oral SAS cell line in the left or right flank to form xenograft tumors. The xenograft tumors were allowed to grow for 14 days to a volume of 27 mm³ (tumor size=a*a*b/2, a: lesser dimension, b: greater dimension) and then were injected daily for 13 days with 10 ul of either PBS ((-▪-) in FIG. 11 and also referring to FIG. 12D) or the KZ-sarcin ((--) in FIG. 11 and also referring to FIG. 12B). Tumor size was measured daily. A mixed model analysis was performed to evaluate the difference in incremental growth ratio between two groups over time using the SAS/STAT MIXED procedure for Windows version 9.1. At the end of 2 weeks, the animals were euthanized, and the tumors were excised and immersed in 10% formalin for tissue sectioning and processed for H&E staining (FIG. 13). FIG. 11 and FIG. 12 show that KZ-sarcin significantly inhibited, or even stopped the tumor growth. Referring to FIG. 13A-13B, after KZ-sarcin treatment, chromatin condensation, multiple nuclear fragmentation and apoptotic bodies were formed. Cells in the periphery tissue around the tumor (without contact with KZ-sarcin (Kazecin)) still maintained normal cell morphology and normal nuclear pattern (FIG. 13C-13D). It means that the recombinant cytotoxin of the present invention could induce a cell into the apoptotic pathway and the toxicity of KZ-sarcin was controlled.

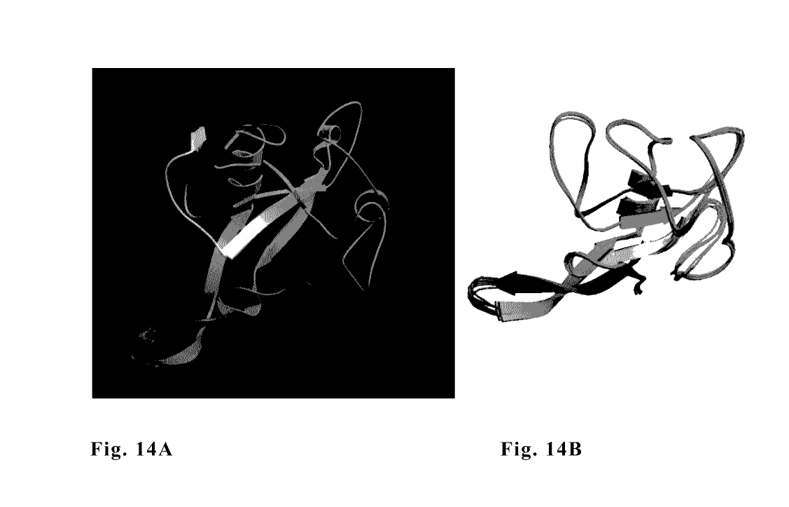

FIG. 14 shows the 3-Dimensional structure of KZ-sarcin of the present invention. In FIG. 14A, the blue region was loop 2 of KZ-sarcin. Compared with the loop 2 (blue part in FIG. 14B) of wild type α-sarcin, the insertion of DEVD sequence did not changed the structure of KZ-sarcin loop 2 of the present invention (FIG. 14A).

As mentioned above, the fungal-originated ribotoxin can inhibit the ribosomal protein synthesis, but cannot easily move into cells. In the present invention, a penetrating peptide such as Tat of HIV, is linked with the fungal-originated cytotoxin using the recombinant DNA technology so that the fungal-originated ribotoxin can penetrate a target cell (e.g., cancer cell) and kill the target cell.

However, the cytotoxin linked with cell penetrating peptide would kill not only the target cell, but also the periphery cells through utilization of conventional and existing techniques. In order to improve the problems, a specific sequence which can be recognized by caspase-3 (e.g., Asp-Glu-Val-Asp (DEVD)) is used to be inserted to the loop region of fungal-originated ribotoxins, particularly loop 2. In addition, the recombinant ribotoxins still maintain the activity of wild-type cytotoxin. Thus, the recombinant cytotoxins of the present invention only enter target cells, and could be cleaved by caspase-3 generated from apoptotic pathway of the target cells. Accordingly, the recombinant cytotoxins, for example but are not limited, KZ-sarcin of the present invention specifically target the cancer cells and do not affect the other normal cells.

In the description above, numerous specific details are set forth. However, it is understood that embodiments of the invention may be practiced without these specific details. For example, well-known equivalent components and elements may be substituted in place of those described herein, and similarly, well-known equivalent techniques may be substituted in place of the particular techniques disclosed. In other instances, well-known structures and techniques have not been shown in detail to avoid obscuring the understanding of this description,

Although the present invention has been described with reference to specific embodiments, this description is not meant to be construed in a limiting sense. Various modifications of the disclosed embodiments, as well as alternative embodiments, will be apparent to persons skilled in the art. It is, therefore, contemplated that the appended claims will cover all modifications that fall within the scope of the present invention.

What is claimed is:

1. A recombinant cytotoxin, comprising
a cytotoxin,
a penetrating peptide linked to the N-terminus or C-terminus of the cytotoxin, and
a Asp-Glu-Val-Asp (DEVD) sequence,
wherein the DEVD sequence is inserted to the cytotoxin.

2. The recombinant cytotoxin of claim 1, wherein the penetrating peptide is linked to the cytotoxin via a linker.

3. The recombinant cytotoxin of claim 1, wherein the cytotoxin comprises ribotoxin, Snake venom, bee venom, Jellyfish venom or toad venom.

4. The recombinant cytotoxin of claim 3, wherein the ribotoxin is selected from a group consisting of fungal-originated ribotoxin, ricin, abrin, emetine, diphtheria toxin, cinnamomin and camphorin.

5. The recombinant cytotoxin of claim 4, wherein the fungal-originated ribotoxin comprises α-sarcin, gigantin, mitogllin, restrictocin, allergen, clavin or tricholin.

6. The recombinant cytotoxin of claim 1, wherein the penetrating peptide comprises Tat, antennapedia or polyarginine.

7. The recombinant cytotoxin of claim 6, wherein the Tat is inserted to the N-terminus of the cytotoxin.

8. The recombinant cytotoxin of claim 1, wherein the DEVD is inserted to the loop region of the ribotoxin.

9. The recombinant cytotoxin of claim 8, wherein the DEVD sequence is inserted to the loop 2 of the ribotoxin.

10. A pharmaceutical composition comprising the recombinant cytotoxin of claim 1 and a pharmaceutically acceptable carrier.

11. Use of the recombinant cytotoxin of claim 10 for preparing a pharmaceutical composition for treatment of cell proliferation disease.

12. The use of claim 11, wherein the recombinant cytotoxin is topically administrated.

13. The use of claim 11, wherein the cell proliferation disease is cancer.

14. The use of claim 13, wherein the cancer comprises oral cancer, breast cancer, prostate cancer, leukemia, colorectal cancer, uterine cancer, ovarian cancer, endometrial cancer, cervical cancer, testicular cancer, lymphoma, rhabdomyosarcoma, neuroblastoma, pancreatic cancer, lung cancer, brain tumors, skin cancer, stomach cancer, liver cancer, kidney cancer or nasopharyngeal cancer.


SEQUENCE LISTING (9 sequences)

SEQ ID NO: 1 - DNA, 50 bp, Artificial Sequence
  N1-primer
nnnccatgggtagaaaaaaacgaagacaacgacgaagaggtggtggtagc

SEQ ID NO: 2 - DNA, 42 bp, Artificial Sequence
  N2-primer
gacgaagaggtggtggtagcgtgacctggacctgcttgaacg

SEQ ID NO: 3 - DNA, 33 bp, Artificial Sequence
  C-primer
taaagcggccgcatgagagcagagcttaagttc

SEQ ID NO: 4 - protein, 11 aa, Artificial Sequence
  The basic domain sequence of Tat peptide
MGRKKRRQRRR

SEQ ID NO: 5 - protein, 4 aa, Artificial Sequence
  linker
GGGS

SEQ ID NO: 6 - DNA, 39 bp, Artificial Sequence
  The upper primer
gacgaagtggatggcaagagtgatcactacctgctggag

SEQ ID NO: 7 - DNA, 21 bp, Artificial Sequence
  The lower primer
cttgctgtgcttgggaggacg

SEQ ID NO: 8 - protein, 4 aa, Artificial Sequence
  DEVD sequence
DEVD

SEQ ID NO: 9 - protein, 198 aa, Artificial Sequence
  KZ-sarcin recombinant protein
MKYLLPTAAAGLLLLAAQPAMAMGRKKRRQRRRGGGSVTWTCLNDQKNPKTNKYETKRLLCNQNKAESNSHHAPLSDGKTGSSYPHWFTNGYDGEGKILKGRTPIKFGKSDCDRPPKHSKDEVDGKSDHYLLEFPTFPDGHDYKFDSKKPKEDPGPARVIYTYPNKVFCGIIAHTKENQGELKLCSHAAALEHHHHHH